(19) United States
(12) Patent Application Publication
(10) Pub. No.: US 2007/0179395 A1
(43) Pub. Date: Aug. 2, 2007

(54) System and method for assessment of sleep

(76) Inventors: John G. Sotos, Palo Alto, CA (US); John L. Branscum JR., Belmont, CA (US)

Correspondence Address: John Sotos, #415, 1788 Oak Creek Dr., Palo Alto, CA, 94304, US

(21) Appl. No.: 11/652,956

(22) Filed: Jan. 12, 2007

Related U.S. Application Data
(63) Continuation-in-part of application No. 11/094,911, filed on Mar. 30, 2005, now abandoned.
(63) Continuation-in-part of application No. 11/095,154, filed on Mar. 31, 2005.
(63) Continuation-in-part of application No. 10/214,792, filed on Aug. 7, 2002.
(60) Provisional application No. 60/557,735, filed on Mar. 30, 2004.
(60) Provisional application No. 60/759,924, filed on Jan. 19, 2006.

(51) Int. Cl. A61B 5/08

(52) U.S. Cl. 600/529

(57) ABSTRACT

A method and system for characterizing breathing in an organism are disclosed. The method acquires data values indicative of periodic respiratory function, for example, tracheal sound envelope data acquired during a period of time associated with a sleep period of the organism. The method defines a first subset of data values among the data values and a second subset of data values among the data values. Cross-correlating the first and second subsets of data values yields a first result vector which is truncated, normalized, and output. These steps are optionally repeated for other subsets of data. From the resulting output, the organism's average respiratory rate may be determined. In addition, periods of time corresponding to sleep, wakefulness, rapid-eye movement (REM) sleep, and non-REM sleep may be identified. The time it takes the organism to fall asleep may also be determined.

### CROSS-REFERENCES TO RELATED APPLICATIONS

This application is a continuation-in-part of application Ser. No. 11/094,911 filed on Mar. 30, 2005. application Ser. No. 11/094,911, in turn, claims priority to U.S. Provisional Patent No. 60/557,735 filed Mar. 30, 2004.

This application is a continuation-in-part of application Ser. No. 11/095,154 filed on Mar. 31, 2005.

This application is a continuation-in-part of application Ser. No. 10/214,792 filed on Aug. 7, 2002.

This application claims priority to U.S. Provisional Patent No. 60/759,924 filed on Jan. 19, 2006, and hereby incorporated by reference for all purposes.

### COPYRIGHT NOTICE

A portion of the disclosure recited in the specification contains material which is subject to copyright protection. Specifically, a source code appendix is included that lists instructions for a process by which the invention is practiced in a computer system. The copyright owner has no objection to the facsimile reproduction of the specification as filed in the Patent and Trademark Office. Otherwise, all copyright rights are reserved. This source code appendix is herein incorporated by reference in its entirety for all purposes.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

The present invention generally relates to ways of assessing the state of an organism. More particularly, the invention provides a method and system for assessing respiratory and sleep related phenomena of an organism. Merely by way of example, the invention is applied to assessing breathing during sleep using one or more sensors and computing hardware. But it would be recognized that the invention has a much broader range of applicability such as applicability cyclic phenomena (e.g. certain heart functions, among others).

Many diseases afflict humans and other organisms. In humans, more than eighty such diseases comprise a class of ills known as sleep disorders. Some sleep disorders pose serious threats to health and well-being, and some are often treatable. Others are believed to be untreatable. As merely an example, sleep disorders are common. Sleep disorders includes, but are not restricted to: snoring, insomnia, restless legs syndrome, narcolepsy, upper airway resistance syndrome (UARS), and sleep apnea and its subtypes: obstructive sleep apnea (OSA) and central sleep apnea (CSA).

To characterize disorders afflicting organisms during sleep, diagnostic tests known as “sleep studies” may be performed. During a typical sleep study, physiological data are collected from the organism by various physiological sensors during a night's sleep. Environmental and other types of data may also be collected. A type of sleep study called polysomnography (PSG) normally collects physiological data from a plurality of data channels over several hours. Belcher (Sleeping: On the Job! 2002, page 138; ) describes 16 to 18 different data channels for a typical PSG study. The resulting data set may be large. Lipman (Snoring from A to Zzzz. 1998, page 115) reports that a paper record of a PSG study may require one-half mile of paper. Computers and digital data storage have, in many cases, reduced the need for paper in sleep studies, but the quantity of information resulting from a sleep study may still tax the patience of a busy human who wants to rapidly assess the implications of the data.

Much of the data collected during a sleep study are quantitative. Sleep studies may yield large amounts of data. Efficiently presenting a large data set to a busy human can be challenging. Presenting quantitative data graphically has often proven advantageous. Tufte (The Display of Quantitative Information. 1983, page 9) notes: “Modem data graphics can do much more than simply substitute for small statistical tables. At their best, graphics are instruments for reasoning about quantitative information. Often the most effective way to describe, explore, and summarize a set of numbers—even a very large set—is to look at pictures of those numbers. Furthermore, of all methods for analyzing and communicating statistical information, well-designed data graphics are usually the simplest and at the same time the most powerful.”

Well-designed data graphics are, of course, generally advantageous, and Tufte has spent considerable effort teaching the principles of good data graphical design. He believes (Tufte. Supra. Page 13) graphical displays should, among other desiderata: show the data; avoid distorting what the data have to say; present many numbers in a small place; make large data sets coherent; encourage the eye to compare different pieces of data; and reveal the data at several levels of detail, from a broad overview to the fine.

Data from sleep studies have been displayed in a plurality of graphical formats, often satisfying Tufte's desiderata only partially. FIGS. 1A and 1B (from Undevia et al. Internet document, 2004) shows approaches typical of the current art.

FIG. 1A shows approximately 5 minutes of data from a polysomnogram. Information from at least 17 channels of data are graphically presented. Because a PSG study may collect data for 8 hours or longer, on the order of 100 such pages may be required to fully present a single study. Many humans find that reviewing 100 pages demands an inconveniently large amount of time.

FIG. 1B shows approximately 6 hours 43 minutes of four data channels from a PSG study, plotted in four separate panes (from Undevia et al. Supra). From top to bottom the panes plot sleep stage, oxygen saturation, apnea-hypopnea event types, and delivered facemask pressure against time. Some of these data inherently vary slowly, allowing longer periods of time to be plotted in a given space with adequate resolution. Plotting certain types of data, e.g. electrocardiogram (EKG) signals, at the time scale of FIG. 1B would typically be far less informative because such data signals inherently vary faster: the spatial resolution of a typical display would not be fine enough to resolve each heartbeat's EKG complex.

Because breathing is a concern in several sleep disorders (including, but not restricted to, sleep apnea and snoring), data about respiration may be usefully presented to a human. Among the respiration-related data sources present in FIG. 1A, for example, are the channels related to airflow, chest and abdominal motion, as well as arterial oxygen saturation (SAO2).

FIG. 1B shows approximately 6 hours 43 minutes of four data channels from a PSG study, plotted in four separate panes (from Undevia et al. Supra). From top to bottom the panes plot sleep stage, oxygen saturation, apnea-hypopnea event types, and delivered facemask pressure against time. Some of these data inherently vary slowly, allowing longer periods of time to be plotted in a given space without losing resolution. Plotting certain types of data, e.g. electrocardiogram signals, at the time scale of FIG. 1B would typically be far less informative because such data signals inherently vary faster. Although FIG. 1B needs only one page to plot results from the entire time of a sleep study, it appears to have a lower information density than FIG. 1A.

Despite the high density of information in FIG. 1A and the long period of time plotted in FIG. 1B, there are many parameters of sleep that are not presented. Some of these parameters are of clinical significance. In particular, parameters which cannot faithfully be reduced to a single value varying over time are problematic to present in the frameworks out FIGS. 1A and FIGS. 1B. Such a limitation can also limit the information that a human health care professional can extract from a sleep study.

From the above, it is desirable to have improved techniques for monitoring health related disorders.

### BRIEF SUMMARY OF THE INVENTION

The present invention generally relates to ways of monitoring health related disorders. A method and system for characterizing breathing in an organism are disclosed. The method acquires data values indicative of periodic respiratory function, for example, tracheal sound envelope data acquired during a period of time associated with a sleep period of the organism. The method defines a first subset of data values among the data values and a second subset of data values among the data values. Cross-correlating the first and second subsets of data values yields a first result vector which is truncated, normalized, and output. These steps are optionally repeated for other subsets of data.

In an embodiment of the invention, one-minute blocks of tracheal sound envelope data are auto-correlated to yield a correlation vector. The vector is truncated to remove values associated with negative lag times. The vector is further truncated to remove lag times associated with correlations faster than a reasonable upper limit for respirations in a human outside of a hospital (e.g. 50/minute). The vector may be further truncated to remove values associated with a lag time corresponding to events occurring with a periodicity (e.g. 5/min) longer than a reasonable lower limit for respiratory rate (and low-order harmonics) in humans.

In an embodiment of the invention, the truncated vector is smoothed and then normalized according to the value of the largest value in the smoothed vector. A plurality of vectors is plotted against a time axis in a format called the “respiratory correlogram” (RCG). Other parameters may be plotted against the time axis.

From inspection of the resulting RCG, the organism's average respiratory rate may be determined. In addition, periods of time corresponding to sleep, wakefulness, rapid-eye movement (REM) sleep, and non-REM sleep may be identified. Brief awakenings and the time it takes the organism to fall asleep may also be determined.

### BRIEF DESCRIPTION OF THE DRAWINGS

FIGS. 1A and 1B illustrates a visualization of polysomnographic information.

FIG. 2 shows a method of the present invention.

FIG. 3 shows truncation and normalization of a correlation vector.

FIG. 4 shows a respiratory correlogram (RCG).

FIG. 5 shows an RCG and hypnogram in a subject with wakefulness and sleep.

FIG. 6 shows an RCG and hypnogram in a subject with wakefulness, rapid eye movement (REM) sleep, and non-REM sleep.

FIG. 7 shows an RCG and hypnogram in a subject with predominant wakefulness.

FIG. 8 shows an RCG and hypnogram in a subject with obstructive sleep apnea.

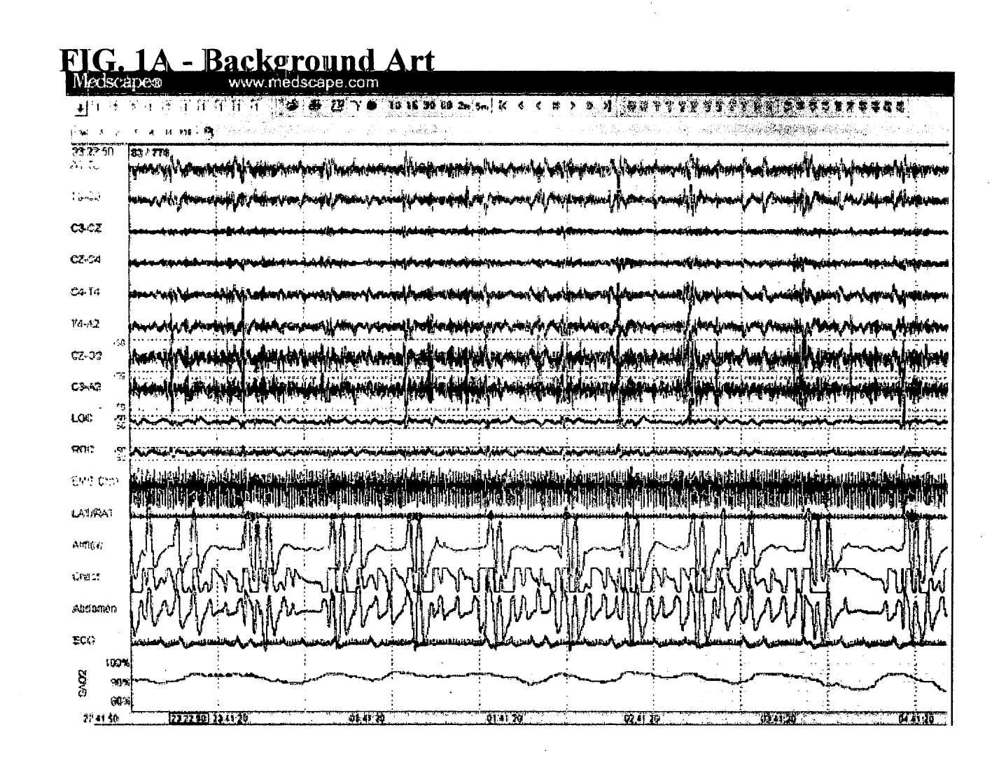

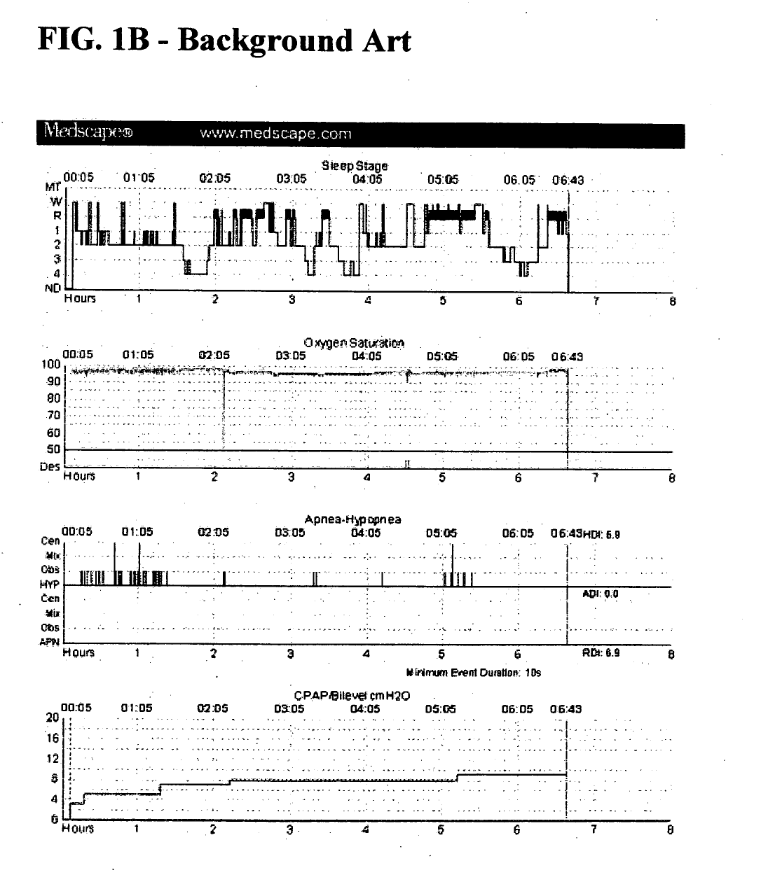

### DETAILED DESCRIPTION OF THE INVENTION

The present invention generally relates to ways of assessing the state of an organism. More particularly, the invention provides a method and system for assessing respiratory and sleep related phenomena of an organism. Merely by way of example, the invention is applied to assessing breathing during sleep using one or more sensors and computing hardware. But it would be recognized that the invention has a much broader range of applicability, such as applicability to cyclic phenomena (e.g. certain heart functions, among others).

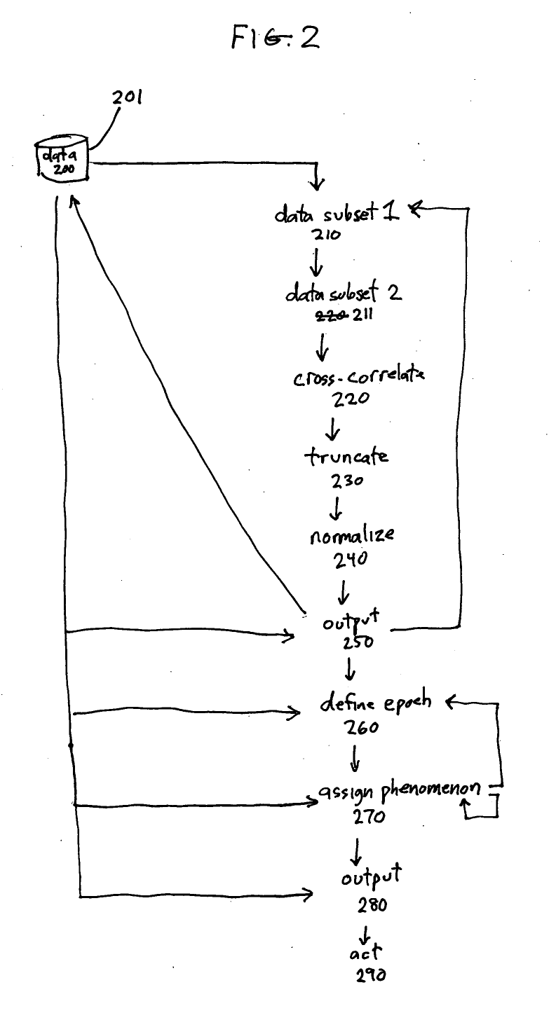

FIG. 2 shows an embodiment of the present invention. Memory 201 contains data 200 obtained from an organism or the organism's environment. In general, data 200 is expected to reflect a cyclical phenomenon (or phenomena), but there is no requirement that this be so. For example, respiration is generally a cyclical phenomenon, as is contraction of the heart, as are peristaltic contractions, as are various functions labeled “circadian” (e.g. sleep), “ultradian,” and so forth. Some cyclical phenomena may have complex periodicity, e.g. the classically described tendency to sleep, which appears to have more than one peak per day, of different sizes.

In an embodiment, data 200 includes tracheal sound recordings made by a tracheal microphone and recorded on an iPod. In an embodiment, data 200 includes tracheal sound recordings obtained from an organism via the system or method disclosed in co-pending U.S. patent application Ser. Nos. 10/721,115 and 11/094,911. Alternatively, an embodiment may include sound data that has been processed in data 200. For example, tracheal sound data having a sampling rate of approximately 2000 hertz may be filtered and rectified. (We have found that a high-pass filter of 400 hertz satisfactory to remove many non-respiratory sounds from tracheal sound data.) We call the resulting rectified data the “envelope” or the “tracheal sound envelope.” Computation of a tracheal sound envelope may be performed by a computer (not shown) having read/write access to memory 200 or a portion of memory 200. In an embodiment, a tracheal sound envelope resulting from a 2000 hertz sound recording is processed by a computer into a tracheal sound envelope having a lower sample rate, e.g. approximately 50 hertz.

In an embodiment, elements of data 200 may be indicative of an organism's respiratory effort, respiratory airflow, exhaled carbon dioxide concentration, snoring sound, cardiovascular sound, bio-electrical potentials (e.g. electrocardiographic potentials), etc.

In an embodiment, memory 201 is digital memory. No limitation is placed on whether memory 201 is volatile or non-volatile. Thus, memory 201 may be, but is not restricted to, random access memory, flash memory, a hard disk, etc. Data 200 may reach memory 201 according to co-pending U.S. patent application Ser. No. 10/214,792.

A subset 210 of data 200 is selected. A subset 211 of data 200 is selected. For example, in an embodiment where data 200 includes tracheal sound envelope data derived from 8 hours of tracheal sound data, subset 210 of data 200 may be the data corresponding to minute 137 of the tracheal sound envelope, and subset 211 of data 200 may be the data corresponding to minute 138 of the tracheal sound envelope.

In general, subset 210 and subset 211 may be selected in either order. In an embodiment, subset 210 and subset 211 may be the same, e.g. when both subsets represent minute 137 of a tracheal sound envelope. In an embodiment, subset 210 may be the same as data 200.

Subset of data 210 and subset of data 211 are cross-correlated 220. In cases where subset 210 and subset 211 are the same, cross-correlating 220 may be appropriately labeled “auto-correlating.” Cross-correlation (or auto-correlation) is well known to persons skilled in the art. Certain software packages, such as Matlab (Mathworks, Inc.) and Octave include subroutines for computing cross-correlations. In an embodiment, subset of data 210 and subset of data 211 are vectors of numbers, and are passed to the Matlab cross-correlation function “xcorr”. (We use the term “vector” to mean a one-dimensional array.) In an embodiment where subset of data 210 and subset of data 211 are the same, a single vector, representing both subsets, is passed to the Matlab auto-correlation function. Henceforth we will usually speak only of cross-correlating, but it should be understood to include auto-correlating when subset of data 210 is the same as subset of data 211.

By way of explanation, cross-correlation generally compares two vectors of numbers according to various lag times. That is, there is a cross-correlation value associated with each of several possible lag times between the two vectors. The resulting cross-correlation values are generally collected in a vector, which we call the “correlation vector,” where each value in the vector may be associated with a different lag time, according to its position in the correlation vector. Typically, the first element in the correlation vector is associated with a lag time of zero seconds.

As is generally known to persons skilled in the art, a high auto-correlation value associated with a lag time of X seconds may be loosely interpreted to mean that, the value the source data at time T is highly correlated with the source data value at time T+X, T−X, and so on for various harmonics. Lag times may be positive or negative. For auto-correlation, the correlation vector is symmetric about a lag time of zero and the correlation value is maximum at a lag time of zero.

The correlation vector that results from cross-correlation 220 is truncated 230. Truncation 230 is generally tailored to characteristics of data subsets 210 and 211 and to the uses to which the invention is being applied. Henceforth, the term “TSE embodiment” will refer to an embodiment where data subsets 210 and 211 are the same, where data subsets 210 and 211 include tracheal sound envelope data, and where issues related to breathing rate and/or regularity are being addressed.

Truncation 230 is optional, however, we have found it beneficial in a TSE embodiment. In such an embodiment, truncation 230 may include discarding correlation values for negative lag times because they are symmetric with values at positive lag times.

In a TSE embodiment truncation 230 may further include discarding correlation values at extreme non-negative lag times. Given a truncation that discards values having a lag time less than X seconds or more than Y seconds, such truncation is metaphorically akin to saying “I am not interested in phenomena (including harmonics) in the tracheal sound envelope signal having a periodicity less than X seconds or more than Y seconds.” For example, in a situation where respiratory rate is being assessed, a reasonable assumption to make is that a human patient will not have a respiratory rate over 50/minute, which would correspond to a lag time of 1/50=0.02 minutes. Setting X to 0.02 minutes in this example would remove the high correlation values at zero lag time and the high correlation values that often occur following the zero lag time point.

Truncation 230 may be implemented in software by techniques known to persons skilled in the art, e.g. by shortening the correlation vector (i.e. by removing elements), or by setting the truncated part of the correlation vector to a special value, or by conducting selected subsequent operations only on the portion of the correlation vector not truncated, and so forth.

The [truncated] correlation vector resulting from step 230 is normalized 240. Normalization 240 involves scaling the values of a correlation vector. In a TSE embodiment, normalization 240 may proceed by first smoothing the correlation vector. Smoothing methods are known to persons skilled in the art. Normalization may then proceed by locating the largest correlation value in the smoothed vector, then dividing all elements in the smoothed correlation vector by the aforementioned largest correlation value. This yields a correlation vector with values ranging potentially from 0 to 1. Other normalization procedures are possible.

Normalization 240 is helpful because the magnitudes of the correlation values depend on the magnitudes of the data in subsets 210 and 211. In a TSE embodiment, for example, tracheal sound associated with normal respirations during sleep may be loud or soft, the volume depending on factors extrinsic to the organism's airway, such as microphone type, distance from microphone to trachea, and so on. Sound volume, is therefore, often a confounding factor in a TSE embodiment, and normalization 240 lessens its contribution to subsequent assessment.

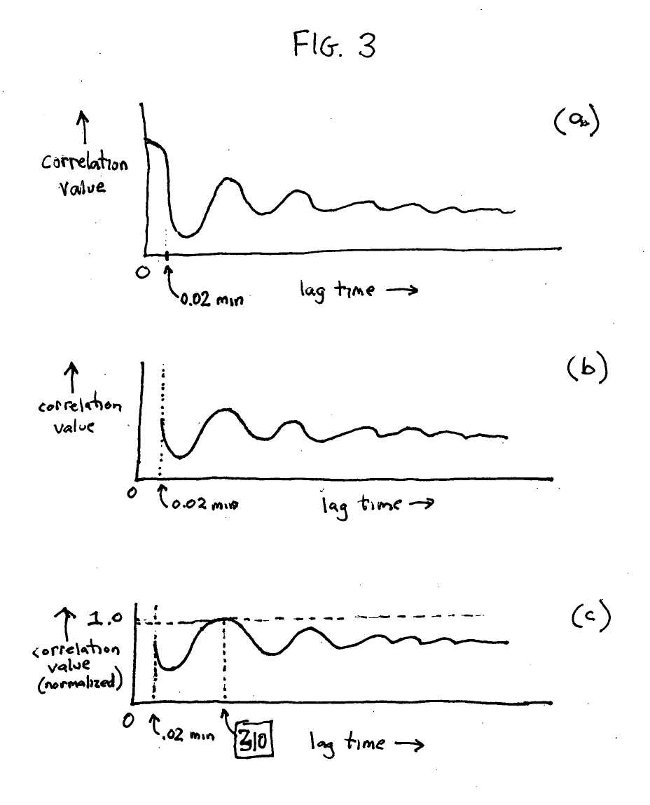

FIG. 3 illustrates the combined effect of truncation and normalization. FIG. 3a plots an exemplary auto-correlation vector against a lag time axis. It is seen that the highest correlation value occurs at lag time zero. FIG. 3b plots the exemplary auto-correlation vector that results after truncation 230 discards values associated with a lag time less than 0.02 minutes. As a result of truncation 230, the largest remaining auto-correlation value is at the time indicated by 310 in FIG. 3c. The correlation value at this time is used as the denominator for normalization 240. We have found that the time 310 of this now-highest correlation value is often indicative of the organism's respiratory rate—a quantity that is often of physiological interest.

Current value of the correlation vector is output 250. In an embodiment, output 250 may include writing the value to memory 200. In an embodiment, output 250 may be directed to a medium accessible to human senses, including, but not restricted to, conversion to a visual form displayed on paper or a computer screen or other visual output device, conversion to a sonic form for listening, conversion to a tactile form for touching, and so on.

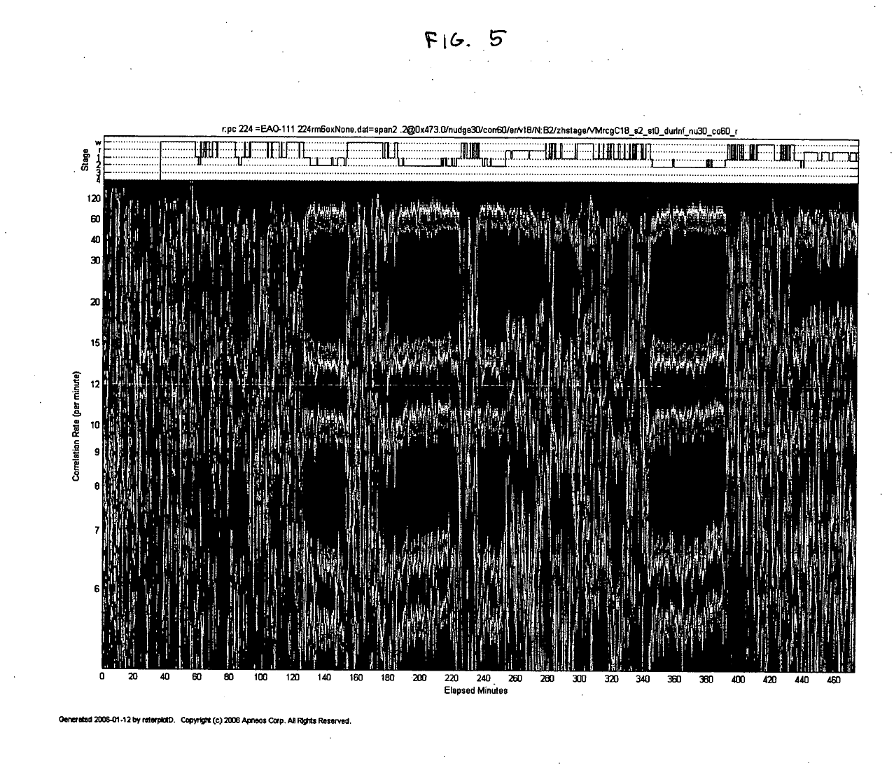

Output 250 may also include data 200 that has not been subjected to steps 210-250. For example, correlation results for tracheal sound envelope data may be visually displayed on a time axis against which oxygen saturation, arm movement, sleep/wake stage, body position, arterial tone or diameter, or other parameter(s) are also displayed. Data plotted in conjunction with output 250 may or may not derive from data 200 in memory 201. FIG. 5, discussed later, is an example in which a hypnogram is plotted against the same time axis as output 250.

The sequence of steps 210-250 may be repeated for other subsets of data 200 from memory 201. In general, the parameters governing iteration of steps 210-250 are determined empirically, and depend on the nature of the underlying data, and the type of assessment(s) to be made. In a TSE embodiment, we have obtained superior results by iterating through tracheal sound envelope data in memory 201 one minute at a time such that each iteration begins 30 seconds later than the previous iteration.

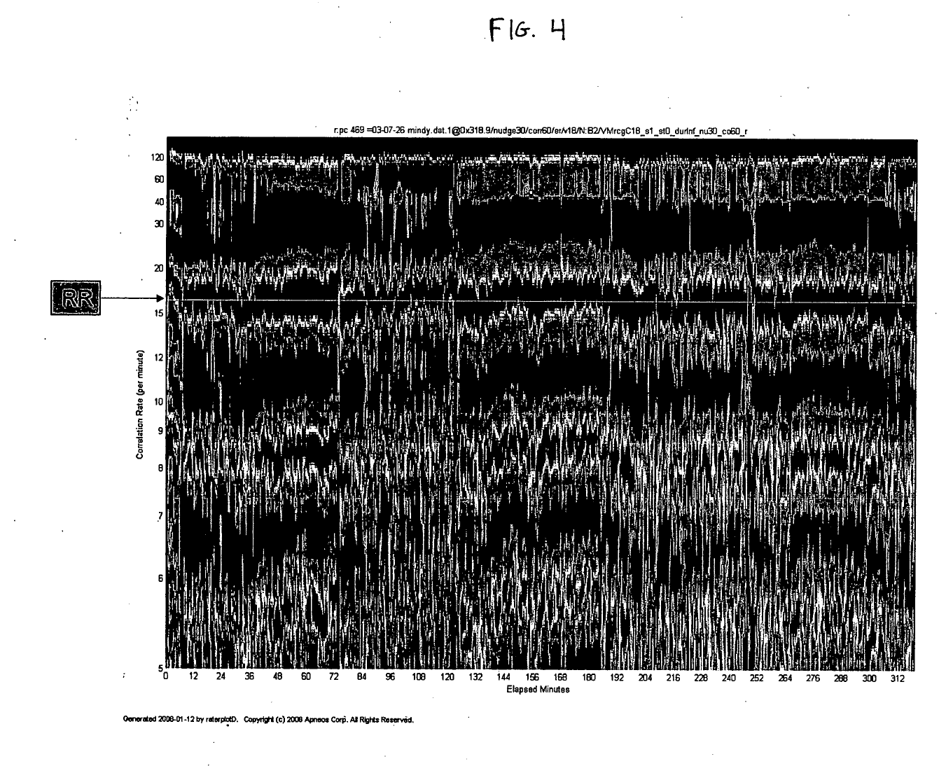

The combined values of iterations through steps 210-250 may be displayed. For example, FIG. 4 plots auto-correlation vectors against a time axis. Each auto-correlation vector resulting is output 250 as a linear, multi-colored vertical region. The vertical axis is labeled “correlation rate,” which is the reciprocal of lag time; the scale of this axis in FIG. 4 is non-linear. In the coloring scheme used in FIG. 4 (and other figures herein), red indicates the highest correlation values and blue the lowest.

FIG. 4, which we call a “respiratory correlogram” (RCG) was generated by a TSE embodiment using parameter values mentioned above for filtering and iteration. Truncation occurred at lag times corresponding to rates less than 5/minute and at lag times corresponding to rates faster than 48/minute. Normalization proceeded as described. Other RCG plots herein were similarly generated from data obtained from human subjects. We have discovered that, in most persons who have normal sleep breathing, a predominant “average” respiratory rate can be identified by visually inspecting RCG plots. In FIG. 4, for example, there is a clear band of red at a lag time corresponding to approximately 17 events per minute.

Given a plurality of correlation vectors that have been output 250, assessment of the organism may proceed by using the correlation vectors to identify an epoch of time 260 and assigning to that epoch a probability that a certain phenomenon (physiological or otherwise) has occurred 270 during that epoch.

For example, FIG. 5 shows an RCG 510 plotted on the same time axis as a hypnogram 520 (i.e. a plot of sleep/wake stage). To a human eye, RCG 510 in FIG. 5 immediately presents several distinctive sub-regions, based on visual appearance. These regions can be selected as an epoch of interest, as per step 260. For example, the color patterns in each vertical line from approximately minutes 120 to 155 appear “organized” or similar to each other (but not, obviously, identical in all cases). This could define one epoch. By contrast, the color patterns in the vertical lines from minutes 0 to approximately 120 appear “disorganized” or markedly different from each other (though not in all cases). This could define another epoch.

We have discovered that an RCG's appearance may be a visual indicator of breathing regularity by an organism. An “organized” RCG segment is an indicator of regular respirations. A “disorganized” RCG segment is an indicator of irregular respirations. Thus, in FIG. 5, minutes 0 to approximately 120 correspond to less regular breathing patterns, while minutes from (approximately) 120 to 155 correspond to more regular breathing patterns. These conclusions would correspond to step 270, e.g. a high probability of the phenomenon “regular respirations” is assigned to the epoch from 120 to 155 minutes (approximately) and a high probability of “irregular respirations” is assigned to the epoch from 0 to approximately 120 minutes.

Multiple epochs may be defined. More than one phenomenon may have a probability assigned per epoch. Epochs may overlap. Epochs do not necessarily have uniform duration. Epoch definition 260 and phenomenon assignment 270 may employ data 200 that has not been subjected to steps 210-250. For example, epoch definition 260 and phenomenon assignment 270 may employ a record of the organism's arm movements during the period of time corresponding to the iterations through steps 210-250. Assigning probabilities of 0 or 1 are the same as no/yes decisions about whether a phenomenon is present during an epoch.

The result of assigning 270 a phenomenon and a probability to an epoch 260 is ultimately output 290. Output 290 could be in the form of a printed report, meant to be read by a human (e.g. a physician or other health care professional, or by the subject organism). Output 290 could be in the form of a record written into a computer database. Output 290 could be in the form of a multimedia presentation that includes elements of data 200 (or other data), e.g. provides audio playback of tracheal breath sounds with annotations indicating phenomena that are present (e.g. have probability above a certain threshold) during various epochs. Other output 290 forms are possible.

The present invention has additional embodiments.

Mathematical tools may be used to characterize and identify “organized” and “disorganized” sets of correlation vectors. For example, the similarity of one correlation vector with another may be expressed by techniques known to persons skilled in the art, such as the cosine distance, or by simple vector subtraction, or by the standard deviation of the vector difference. Other methods are known to persons with ordinary skill in the art.

In some cases, it is beneficial to compare a correlation vector to a known template. For example, normal respirations may be identified as having a stereotypical correlation vector. To the extent that a given correlation vector differs from such a stereotyped template, it (and the time it represents) may be characterized as atypical for quiet respiration.

For example, in FIG. 5, hypnogram 520 shows a correlation between wakefulness and a disorganized RCG pattern, and a correlation between sleep and an “organized” pattern. That is, times of disorganized RCG appearance tend to occur during or close to times the hypnogram indicates as wakefulness, while times of organized RCG appearance tend to occur during or close to times the hypnogram indicates as sleep. The correlation is not perfect.

It is known to persons skilled in the art that sleeping is sometimes associated with relatively regular breathing. It is further known to such persons that, as a rule of thumb, breathing during quiet sleep may be more regular than breathing during active wakefulness.

We have discovered that the RCG appearance may in some cases make visible the relative respiratory regularity of breathing while asleep. Thus, in some cases an RCG's appearance may be employed to determine whether an organism is awake or asleep during particular times, given the differences in respiratory regularity during sleep and wakefulness.

FIG. 5 includes “organized” and “disorganized” regions of correlation vectors that may be identified by a human eye and brain. Mathematical techniques may also be used. Such techniques might be based on techniques that, as noted, quantify organization and disorganization. An examplary technique is the method of Foote, used to identify segments of music; it can also be applied to correlation vectors or to envelopes (and other signals) derived from tracheal sound.

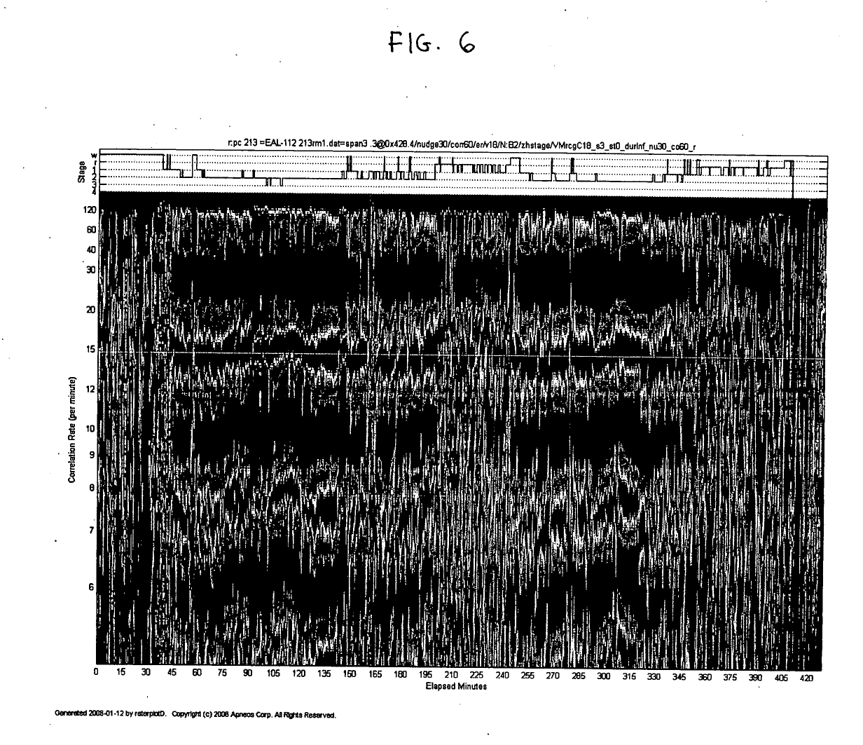

FIG. 6 contains a plot of an RCG 610 and a hypnogram 620 from a human subject. In this figure, three levels of organization may be discerned among the correlation vectors plotted in RCG 610. From minute 0 to minute 30, the correlation vectors appear disorganized. From minute 45 to minute 200, RCG 610 appears organized. From minute 200 to minute 230, however, RCG 610 appears organized to a degree between the degrees of organization in the other two epochs noted. (All times are approximate.) Hypnogram 620 shows that the minutes 0-45 are generally wakefulness, minutes 45-200 are generally stage 1 or stage 2 sleep, and that minutes 200-230 are generally rapid eye movement (REM) sleep. From the aforementioned, it is apparent that in come cases an RCG can be used to infer when a patient is in REM sleep and when a patient is in a state of sleep other than REM sleep, by identifying three degrees of organization of correlation vectors, and assigning wakefulness, REM sleep, and non-REM sleep to the degrees (from most disorganized to least disorganized). Non-REM sleep includes stages 3 and 4 sleep as well as stages 1 and 2 sleep.

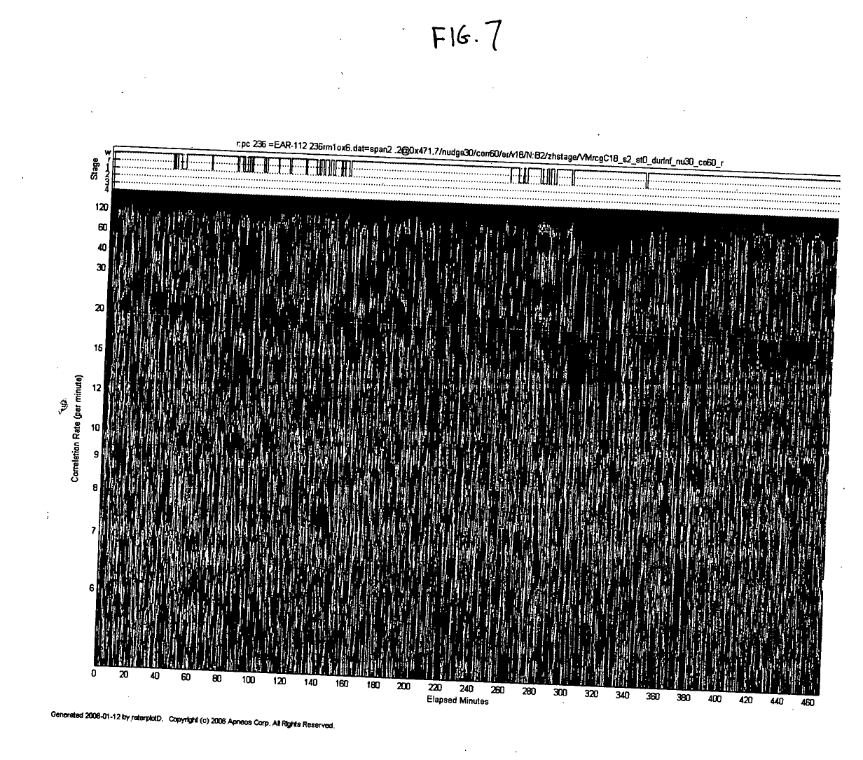

FIG. 7 shows an example of an RCG and hypnogram from a human subject who slept very little during their sleep study. The disorganization of the RCG is apparent to the human eye. Nevertheless, a predominant respiratory rate can be identified, and is indicated by a horizontal line at approximately 20/minute. FIGS. 4, 5, 6, and 8 also show horizontal lines indicating the “average” respiratory rate during the duration of time covered by the RCG. “Average” is used in quotes because the word is not used as a synonym to “mean.”

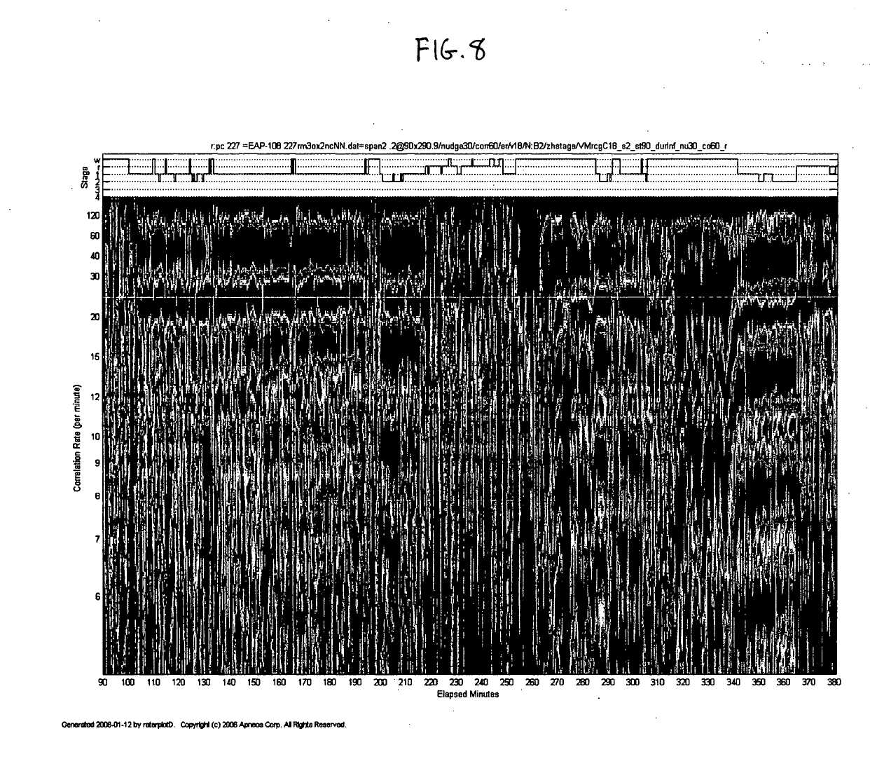

FIG. 8 shows and RCG and hypnogram from a human subject who has obstructive sleep apnea. During the epoch from 220 to 250 minutes (approximately), the RCG appears disorganized. The subject during this epoch was experiencing frequent apneic and hypopneic events (not shown). It is apparent that these events can convert the “organized” RCG pattern associated with normal sleep into a disorganized pattern more closely resembling that of wakefulness. Thus, in an embodiment of the present invention, epochs of abnormal sleep breathing are identified and not assigned a probability of sleep or wakefulness (or REM or non-REM sleep) based solely on RCG [dis] organization.

Given the present invention's ability to identify sleep and wakefulness, it is also able to identify time-to-sleep. For example, if the subject triggers the start of data collection at the time he or she (figuratively) turns out the lights and attempts to sleep, then the time from this event to the time where sleep appears in the RCG may be taken as the time it took the subject to fall asleep.

We have discovered that an additional feature of the RCG can increase the accuracy of determining the time-to-sleep. In some subjects, breathing becomes regular while they are relaxed, but awake. This regularization of breathing may be apparent on RCG and may underestimate the time-to-sleep. However, we have discovered that such awake-regularization leading to sleep may also be accompanied by a decline in respiratory rate. Thus, a more accurate estimation of sleep onset can be yielded by finding the start of an RCG epoch which has an organized appearance and also has an “average” respiratory rate that is substantially the same as the “average” respiratory rate during epochs associated with sleep.

Brief awakenings may be identified on the RCG by noting brief epochs of disorganization.

An advantage of the present invention is that sleep/wake state may be determined without measuring electroencephalographic (EEG) signals. Measuring EEG signals often adds complexity to a sleep assessment system or method because, in general, multiple, carefully located sensor leads must be placed on or near an organism's scalp. In general, a system which purports to assess sleep in an organism benefits by actually being able to demonstrate that the organism slept during the period of assessment. Thus, a further advantage of the present invention is that it offers a method of demonstrating that sleep occurred that does not require EEG leads on the subject.

The invention may be applied to data collected from an organism during periods unassociated with sleep. It may, therefore, be applied to data collected at any time. The resulting output is most easily interpreted when data collection occurred when the organism was in a stable state. When assessing respirations, for example, tracheal sound data may be collected from an awake human who is resting comfortably. In this way, an awake respiratory rate can be determined.

A human who is exercising may also supply data, so that an exercise respiratory rate can be determined. The respiratory rate could be computed at various levels of exercise intensity, thereby quantifying the respiratory response to exercise. A maximal respiratory rate, defined as that associated with a subject's peak level of exercise, could also be determined. Serial determinations of a maximal respiratory rate could be used to monitor fitness and health over time, among other phenomena. Subjects with unusual patterns of respiration during exercise could also be identified with the present invention, resulting in the ability to coach them on their breathing technique.

The invention could be used to determine a respiratory rate in various situations, for example, while sleeping, while recumbent, while exercising, or while sitting quietly. These rates could be compared so that, for example, a subject with a recumbent rate significantly higher than a sitting rate might be suspected of displaying orthopnea or other conditions where dyspnea is associated with recumbency. The invention may provide evidence that an apparently asymptomatic subject is affected with such a condition. In this way, the invention could help detect disease earlier.

As noted, the present invention has applicability in patients with illness. Management of patients with cardiopulmonary disorders could usefully employ the present invention when applied to respiration. For example, patients with heart failure may have one or more of tachypnea, paroxysmal nocturnal dyspnea, central sleep apnea, Cheyne-Stokes respiration, orthopnea, all of which classically include altered respiratory patterns.

The present invention could be applied to other disorders with prominent nocturnal respiratory components, e.g. asthma (especially nocturnal asthma), chronic obstructive lung disease, and paroxysmal nocturnal hemoglobinuria.

Some persons with narcolepsy display a “SOREMP”—a period of REM sleep near the time of sleep onset. Because the present invention can be used to identify sleep onset and REM sleep, it may be possible to identify persons with narcolepsy (and other conditions that include a SOREMP) with the present invention.

There present invention may be applied to functions other than respiratory functions. Various functions in living organisms occur cyclically. Respiration and cardiac contraction have been noted. Peristaltic contractions in the digestive system occur cyclically. Certain hormones are secreted in a pulsatile, cyclic pattern (e.g. some gonadotrophic hormones, melatonin). Many functions in living organisms have a circadian cycle, including sleep, body temperature, and so on. With appropriate sensors and data sets, possibly obtained under conditions of interest, these phenomena, too, are amenable to assessment via the present invention.

It should be noted that the above sequence of steps is merely illustrative. The steps can be performed using computer software or hardware or a combination of hardware and software. Any of the above steps can also be separated or be combined, depending upon the embodiment. In some cases, the steps can also be changed in order without limiting the scope of the invention claimed herein. One of ordinary skill in the art would recognize many other variations, modifications, and alternatives. It is also understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A method for characterizing breathing in an organism, the method including:
acquiring data values indicative of periodic respiratory function, the respiratory function being associated with a sleep period of the organism;
defining a first subset of data values among the data values;
defining a second subset of data values among the data values;
cross-correlating the first and second subsets of data values, yielding a first result vector;
truncating the first result vector, yielding a second result vector;
normalizing the second result vector, yielding a third result vector;
outputting the third result vector;
optionally repeating the defining, cross-correlating, truncating, normalizing, and outputting;
